(19) United States
(12) Patent Application Publication
(10) Pub. No.: US 2017/0202433 A1
(43) Pub. Date: Jul. 20, 2017

(54) WIRELESS VISIBLE BOUGIE BASED ON CLOUD TRANSMISSION

(71) Applicant: DEX MEDICAL DEVICE CO., LTD., New Taipei City (TW)

(72) Inventor: CHIEN-LI CHANG, NEW TAIPEI CITY (TW)

(21) Appl. No.: 15/343,235

(22) Filed: Nov. 4, 2016

(30) Foreign Application Priority Data
Jan. 14, 2016 (TW) 105200538

(51) Int. Cl. A61B 1/00; A61B 1/267; G02B 23/24; H04L 29/08; H04N 5/225

(52) U.S. Cl. CPC A61B 1/00016; H04L 67/1097; H04N 5/2254; G02B 23/2484; A61B 1/267; A61B 1/00163; H04N 2005/2255

(57) ABSTRACT

A wireless visible bougie based on cloud transmission mainly includes a WIFI or 3G/4G communication module and mediation platform server placed on an internet cloud. The WIFI or 3G/4G communication module is connected to and exchanges data with an endpoint of a mediation platform server placed on a cloud of an internet through the internet to store image data captured by the visible bougie, thereby building a database in the mediation platform server of the cloud and then forming data control on the cloud, and allowing relevant personnel to download the data from the cloud for observation so as to achieve the objects of mutual observation and learning.

### (a) TECHNICAL FIELD OF THE INVENTION

The present invention relates to a wireless visible bougie based on cloud transmission, and more particularly to a wireless visible bougie, allowing image data captured by the visible bougie to be stored, data control to be formed on cloud, and the data to be downloaded and viewed by relevant personnel, thereby achieving mutual observation and learning.

### (b) DESCRIPTION OF THE PRIOR ART

General endotracheal intubation visible bougies are divided into bougies having endoscope and observation mirror functions, where endoscopes have a surgical function such as blowing, flushing, cutting or electric burning besides the observation of a human body organ state. But, the observation mirrors are only for observation without the surgical functions of endoscopes. A conventional visible bougie, as FIG. 1 shows, is configured with a soft and transparent endotracheal inner tube 1, in which a flexible bougie 2 is inserted, where the front end of the bougie 2 is configured with a tube body 21, inside which elements such as an optical filter, lens and optical element are configured, and on the rear end of which a connector 31 is configured, with the connector 31 being allowed to insert in a control circuit 3. Upon use, the endotracheal inner tube 1 together with the bougie 2 is inserted in a patient's mouth, and the control circuit 3 is connected to a host computer 4 with a display screen 41. Thereafter, the lens is adapted to capture the image of an organ after the optical element is light up, and image information is then transmitted to the control circuit 3, thereby observing the image on the display screen 41 of the host computer 4. However, although conventional visible bougies allow the immediate image viewing mentioned above, image information cannot be stored in an appropriate, safe place to be controlled, cannot be accessed by relevant personnel, and further cannot be observed and learned mutually by relevant personnel such that observation mirrors are difficult to play higher functions.

### SUMMARY OF THE INVENTION

To design a wireless visible bougie based on cloud transmission, and allow image data captured by lens to be stored and controlled, and the data to be observed and learned mutually by relevant personnel, the present invention is proposed.

The main object of the present invention is to provide a wireless visible bougie based on cloud transmission, storing image data captured by the visible bougie in cloud and then forming data control on the cloud, and allowing relevant personnel to download the data from the cloud for mutual observation and learning.

To achieve the object mentioned above, the present invention proposes a wireless visible bougie based on cloud transmission, mainly including a WIFI or 3G/4G communication module and mediation platform server placed on an internet cloud; the WIFI or 3G/4G communication module is connected to and exchanges data with an endpoint of a mediation platform server placed on a cloud of an internet through the internet to store image data captured by the visible bougie in the mediation platform server, thereby building a database in the mediation platform server of the cloud and then forming data control on the cloud, and allowing relevant personnel to download the data from the cloud for observation so as to achieve the objects of mutual observation and learning.

According to the wireless visible bougie based on cloud transmission mentioned above, the control circuit includes a single chip processer adapted to process image signals captured by the visible bougie, and the processed image signals are then transmitted to the communication module.

### BRIEF DESCRIPTION OF THE DRAWINGS

FIG. 1 is a perspective view of a conventional visible bougie;

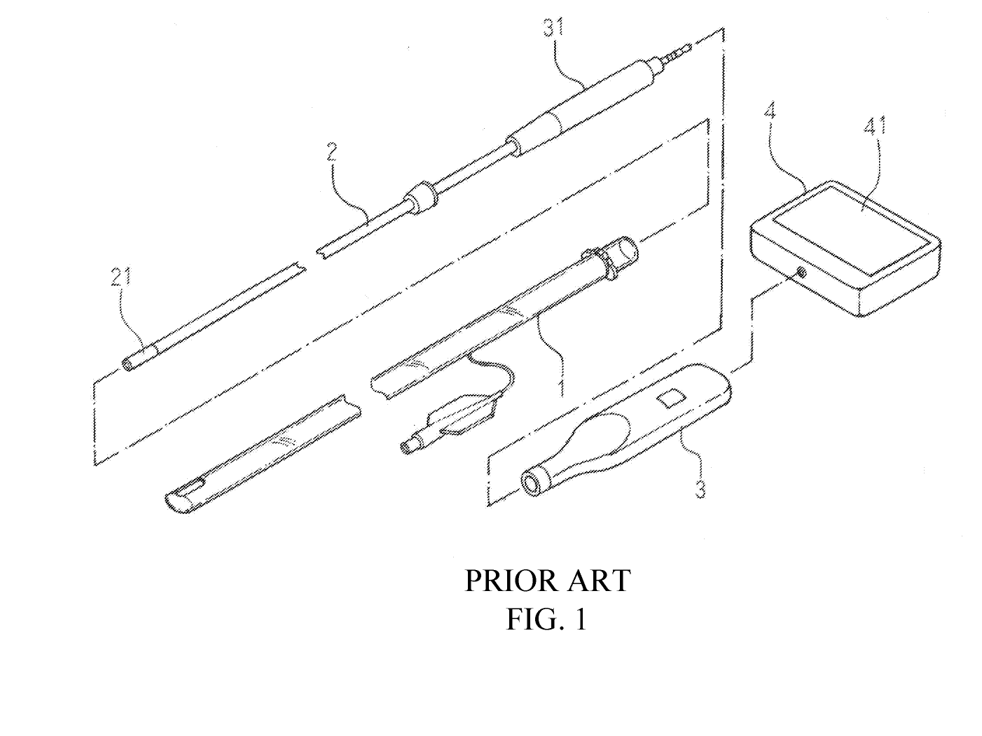

FIG. 2 is a perspective view of a visible bougie according a preferred embodiment of the present invention;

FIG. 3 is a block diagram of the embodiment of the present invention; and

FIG. 4 is perspective view of the embodiment of the present invention in an action state.

### DETAILED DESCRIPTION OF THE PREFERRED EMBODIMENTS

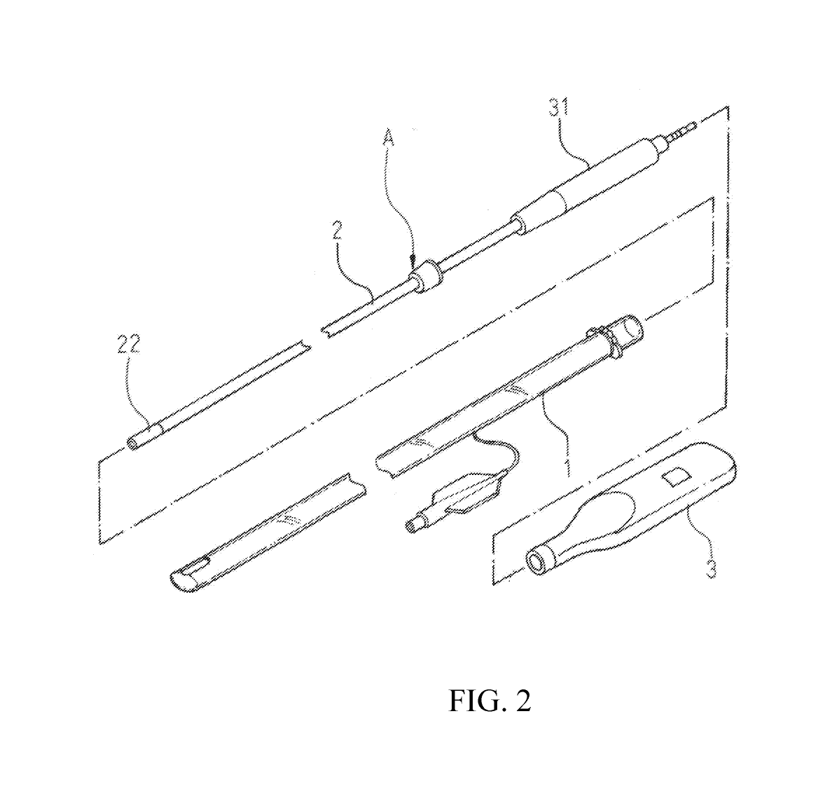

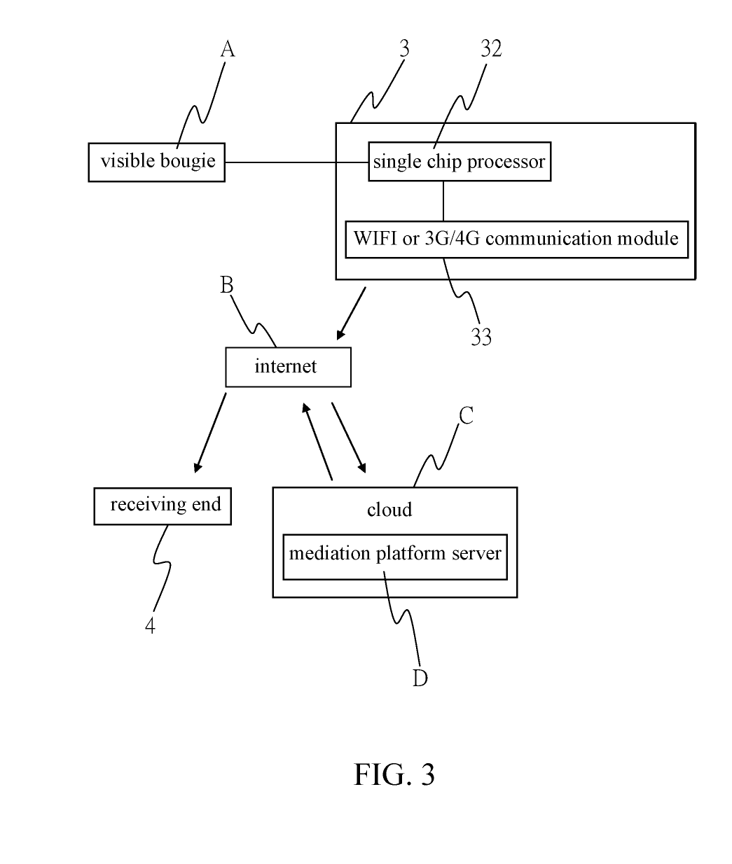

Referring to FIGS. 2 and 3, which respectively are a perspective view and block diagram of a preferred embodiment according to the present invention, a wireless visible bougie based on cloud transmission of the present invention includes a visible bougie A and a mediation platform server D placed on a cloud C of an internet B. As conventional visible bougies, the visible bougie A of the present invention is configured with a soft and transparent endotracheal inner tube 1, in which a flexible bougie 2 is inserted, and a control circuit 3 is configured on the rear end of the bougie 2, where the front end of the bougie 2 is configured with an outer tube body 22, inside which elements such as an optical filter, lens, integrated circuit and flexible circuit board are configured in sequence from front to back, where the flexible circuit board is connected to a connector 31 on the rear end of the bougie 2 for the plugging thereof in the control circuit 3.

The control circuit 3 includes a single chip processor 32 and a WIFI or 3G/4G communication module. Image signals captured by the lens mentioned above are processed through the single chip processor 32, and the processed image signals are then transmitted to the communication module 33.

The wireless visible bougie based on cloud transmission is formed by the combination of the components mentioned above. The wireless visible bougie, when used, can be connected to and exchange data with the endpoint of the mediation platform server D placed on the cloud C of the internet B through the internet B to store the image data captured by the visible bougie A in the mediation platform server D by means of the wireless communication capability of the WIFI or 3G/4G communication module 33 built in the visible bougie A, thereby building a database in the mediation platform server D of the cloud C, and allowing relevant personnel to download the data from the cloud C through a receiving end 4 for viewing, achieving the objects of mutual observation and learning.

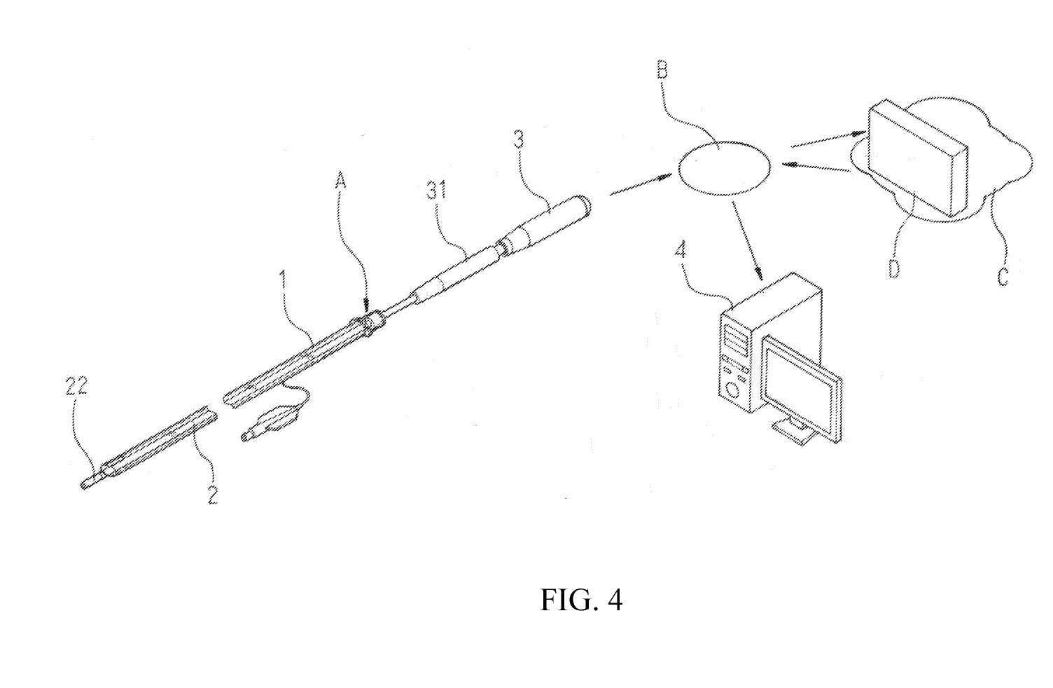

Referring to FIG. 4, which shows the embodiment of the present invention in an action state, and FIGS. 2 and 3 again, the bougie 2, when used, is inserted in the endotracheal inner tube 1, and the endotracheal inner tube 1 is then inserted in a patient's mouth together with the bougie 2. The images of an organ are captured by the lens through the illumination of the LED, and the captured image signals are then transmitted to the control circuit 3 configured on the rear end of the bougie 2. Since the control circuit 3 includes the single chip processer 32 and WIFI or 3G/4G communication module 33, image signals are read and processed by the single chip processor 32 when it receives the image signals, the processed image signals are transmitted to the communication module 33, the communication module 33 is connected to and exchanges the data with the endpoint of the mediation platform server D placed on the cloud C of the internet B through the internet B, and then the mediation platform server D stores the image data captured by the visible bouge A, thereby building a database in the mediation platform D of the cloud C, and then forming data control on the cloud C.

Furthermore, upon a medical surgery, the images of the surgery process captured can be transmitted to the endpoint of the mediation platform server D placed on the cloud C of the internet B by connecting the communication module 33 of the control circuit 3 therewith through the internet B, thereby allowing other medical personnel to observe the surgery process immediately by connecting the receiving end 4 with the endpoint of the mediation platform server D placed on the cloud C through an internet B so as to achieve the objects of mutual observation and learning.

Furthermore, the database is built in the mediation platform server D of the cloud C for storing the data. Thereafter, relevant medical personnel may download the relevant image data from the mediation platform server D of the cloud C by connecting the receiver end 4 with the mediation platform server D of the cloud C through the internet B, thereby achieving the objects mutual observation and learning.

I claim:

1. A wireless visible bougie based on cloud transmission, configuring a soft and transparent endotracheal inner tube, a flexible bougie being inserted in said endotracheal inner tube, a control circuit being configure on a rear end of said bougie, an outer tube being engaged with a front end of said bougie, an optical filter, lens, integrated circuit and flexible circuit board being configured inside said outer tube body in sequence from front to back, and said soft circuit board being in connection with connector on a rear end of said bougie for the insertion thereof in said control circuit, wherein:
said control circuit comprises a WIFI or 3G/4G communication module, said WIFI or 3G/4G communication module is connected to and exchanges data with an endpoint of a mediation platform server placed on a cloud of an internet through said internet to store image data captured by said visible bougie, thereby building a database in said mediation platform server of said cloud and then forming data control on said cloud, and allowing relevant personnel to download said data from said cloud for observation so as to achieve the objects of mutual observation and learning.

2. The wireless visible bougie according to claim 1, wherein said control circuit comprises a single chip processer adapted to process image signals captured by said visible bougie, and said processed image signals are then transmitted to said communication module.
